(19) United States
(12) Patent Application Publication
(10) Pub. No.: US 2018/0011061 A1
(43) Pub. Date: Jan. 11, 2018

(54) OBJECT INFORMATION ACQUIRING APPARATUS AND SIGNAL PROCESSING METHOD

(71) Applicant: CANON KABUSHIKI KAISHA, Tokyo (JP)

(72) Inventor: Yukio Furukawa, Sagamihara-shi (JP)

(21) Appl. No.: 15/546,075

(22) PCT Filed: Feb. 2, 2016
(86) PCT No.: PCT/JP2016/000735

§ 371 Date: Jul. 25, 2017

(30) Foreign Application Priority Data
Feb. 20, 2015 (JP) 2015-032153

(51) Int. Cl. G01N 29/24; A61B 5/00; G01N 29/06

(52) U.S. Cl. CPC G01N 29/2418; G01N 29/0672; A61B 5/0095; G01N 2291/02475

(57) ABSTRACT

an object information acquiring apparatus comprises a light emission unit configured to emit light beams from a plurality of emission positions; a conversion unit configured to convert acoustic waves generated when an object is irradiated with the light beams emitted by the light emission unit into electric signals; a beam profile acquisition unit configured to acquire information relating to beam profiles of the light beams emitted by the light emission unit, the beam profiles corresponding respectively to the plurality of emission positions; and a characteristic information acquisition unit configured to acquire characteristic information of the object on the basis of the information relating to the beam profiles corresponding to the plurality of emission positions and the electric signals.

### TECHNICAL FIELD

The present invention relates to an object information acquiring apparatus.

### BACKGROUND ART

Active research is being conducted in the medical field into optical imaging apparatuses serving as object information acquiring apparatuses that irradiate an object with light beams from a light source such as a laser light source and use photoacoustic waves obtained on the basis of the emitted light beams to form an image from information relating to the interior of the object. One of these optical imaging techniques is Photo Acoustic Tomography (PAT). In PAT, an object is irradiated with a light pulse emitted from a light source, and an acoustic wave generated from tissue that absorbs the energy of the light pulse propagated and diffused in the object is received. A phenomenon whereby a photoacoustic wave is generated is known as a photoacoustic effect, and the acoustic wave generated by the photoacoustic effect is known as a photoacoustic wave. A test segment such as a tumor or a blood vessel is often more highly absorptive to optical energy than tissue on the periphery thereof, and therefore the test segment absorbs a larger amount of light than the peripheral tissue so as to expand momentarily. The photoacoustic wave generated during this expansion is received by an acoustic wave reception element, whereby a reception signal is acquired. By subjecting the reception signal to mathematical analysis processing, a sound pressure distribution of the photoacoustic wave generated by the photoacoustic effect in the interior of the object can be turned into an image (image reconstruction). The image acquired in this image forming process is known as a photoacoustic wave image. An optical characteristic distribution, and more particularly a light absorption coefficient distribution, of the object interior can be acquired on the basis of the photoacoustic wave image. This information can also be used in quantitative measurement of specific substances in the object, such as glucose and hemoglobin contained in blood, for example.

The intensity of the photoacoustic wave or the reception signal obtained therefrom is known to be commensurate with the light absorption coefficient of the generation source and the energy density of the light beam emitted onto the generation source. In other words, when attempting to form an image of the light absorption coefficient distribution of the object interior, it is effective to learn the optical energy distribution of the object interior correctly in order to improve the quantitativity of the light absorption coefficient distribution.

Patent Literature 1 discloses a technique employed in a scanning type photoacoustic imaging apparatus for generating image data from the light absorption coefficient distribution of an object interior on the basis of a beam profile (also referred to as a light intensity profile) of an emitted light beam photographed by an imaging unit and a reception signal of a photoacoustic wave.

### CITATION LIST

### Patent Literature

Patent Literature 1: Japanese Patent Application Publication No. 2011-229756

### SUMMARY OF INVENTION

### Technical Problem

However, it is assumed in Patent Literature 1 that a light intensity distribution of the emitted light beam photographed by the imaging unit does not vary during scanning, and therefore the fact that the beam profile of an emitted light beam emitted from an emission optical system varies according to the scanning position of the emission optical system is not taken into consideration.

Hence, a problem arises in that when the beam profile of the emitted light beam emitted from the emission optical system varies according to the emission position of the emission optical system, the precision with which the light absorption coefficient distribution of the object interior is acquired decreases.

In consideration of this problem, an object of the present invention is to provide an object information acquiring apparatus with which characteristic information relating to an object can be acquired with greater precision.

### Solution to Problem

The present invention in its one aspect provides an object information acquiring apparatus comprising alight emission unit configured to emit light beams from a plurality of emission positions; a conversion unit configured to convert acoustic waves generated when an object is irradiated with the light beams emitted by the light emission unit into electric signals; a beam profile acquisition unit configured to acquire information relating to beam profiles of the light beams emitted by the light emission unit, the beam profiles corresponding respectively to the plurality of emission positions; and a characteristic information acquisition unit configured to acquire characteristic information of the object on the basis of the information relating to the beam profiles corresponding to the plurality of emission positions and the electric signals.

The present invention in its another aspect provides a signal processing method for acquiring characteristic information of an object using electric signals derived from acoustic waves that are generated when the object is irradiated with light beams emitted from a plurality of emission positions, comprising the steps of acquiring information relating to beam profiles of the light beams emitted from the plurality of emission positions, the beam profiles corresponding respectively to the plurality of emission positions; and acquiring the characteristic information on the basis of the information relating to the beam profiles corresponding to the plurality of emission positions and the electric signals.

### Advantageous Effects of Invention

As described above, the present invention provides an object information acquiring apparatus with which characteristic information relating to an object can be acquired with greater precision.

Further features of the present invention will become apparent from the following description of exemplary embodiments with reference to the attached drawings.

### BRIEF DESCRIPTION OF DRAWINGS

FIG. 1 is a block diagram showing a first embodiment of an object information acquiring apparatus according to the present invention.

FIGS. 2A to 2C are pattern diagrams showing rotation of a light intensity profile of a beam section, according to the first embodiment.

FIGS. 3A to 3C are pattern diagrams showing positional deviation in the light intensity profile of the beam section, according to the first embodiment.

FIG. 4 is a pattern diagram showing light emission positions of an emission unit 105 according to the first embodiment.

FIG. 5 is a table showing variation information stored in a variation storage unit according to the first embodiment.

FIG. 6A is a flowchart showing an example of functions of the object information acquiring apparatus according to the first embodiment.

FIG. 6B is a flowchart showing another example of the functions of the object information acquiring apparatus according to the first embodiment.

FIG. 7 is a block diagram showing a second embodiment of the object information acquiring apparatus according to the present invention.

FIG. 8 is a view showing variation in the light intensity profile of the beam section, according to the second embodiment.

FIG. 9 is a table showing calculation results indicating amounts of variation in an emitted light beam, according to the second embodiment.

FIG. 10 is a flowchart showing functions of the object information acquiring apparatus according to the second embodiment.

FIG. 11 is a block diagram showing a third embodiment of the object information acquiring apparatus according to the present invention.

FIG. 12 is a pattern diagram showing setting of an area of interest in the object information acquiring apparatus according to the third embodiment.

FIG. 13 is a flowchart showing functions of the object information acquiring apparatus according to the third embodiment.

FIG. 14 is a block diagram showing a fourth embodiment of the object information acquiring apparatus according to the present invention.

FIG. 15 is a block diagram showing a fifth embodiment of the object information acquiring apparatus according to the present invention.

FIG. 16 is a flowchart showing functions of the object information acquiring apparatus according to the fifth embodiment.

### DESCRIPTION OF EMBODIMENTS

Embodiments of the present invention will be described in detail below with reference to the drawings. Note that in principle, identical reference symbols have been allocated to identical constituent elements, and duplicate description thereof has been omitted. It is to be understood, however, that calculation formulae, calculation procedures, and so on described in detail below may be modified as appropriate in accordance with the configuration of the apparatus to which the invention is applied and various other conditions, and that the scope of the invention is not limited to the following description.

The object information acquiring apparatus according to the present invention includes any apparatus that uses a photoacoustic effect to acquire image data from object information by receiving acoustic waves generated in the interior of an object in response to light beams (electromagnetic waves) such as near-infrared rays emitted onto the object.

In the case of an apparatus that uses the photoacoustic effect, the acquired object information refers to a generation source distribution of an acoustic wave generated in response to an emitted light beam, an initial sound pressure distribution of the object interior, an optical energy absorption density distribution or an absorption coefficient distribution derived from the initial sound pressure distribution, and a concentration distribution of a tissue-forming substance. The substance concentration distribution may be an oxygen saturation distribution, a total hemoglobin concentration distribution, an oxygenated/reduced hemoglobin concentration distribution, and so on, for example.

Further, characteristic information, which is object information acquired in a plurality of positions, may be acquired as a two-dimensional or three-dimensional characteristic distribution. The characteristic distribution may be generated in the form of image data representing the characteristic information of the object interior.

The acoustic wave according to the present invention is typically an ultrasonic wave, but may also be a sound wave or an elastic wave referred to as an ultrasonic wave. An acoustic wave generated by the photoacoustic effect is known as a photoacoustic wave or an optical ultrasonic wave. An acoustic wave reception element (a probe, for example) receives the acoustic wave generated in the object interior.

First, respective constituent elements of an object information acquiring apparatus according to an embodiment of the present invention will be described briefly below.

(Light Source)

When the object is a living organism, a light source serves as means for generating a light beam of a wavelength that is absorbed mainly by a specific component among constituent components of the living organism. The light beam generated by the light source may be a light pulse having a pulse width of approximately 10 to 100 nsec. As a result, a photoacoustic wave can be generated efficiently. The light source is preferably a laser from which a large output is obtained, but the present invention is not limited to this configuration, and a light emitting diode, a flash lamp, or the like may be used instead of a laser. Various lasers, such as a solid-state laser, a gas laser, a dye laser, or a semiconductor laser, can be applied as the laser used as the light source. The wavelength of the light beam generated by the light source is preferably a wavelength at which the light beam propagates to the object interior. When the object is a living organism, the wavelength maybe set at no less than 500 nm and no more than 1200 nm, for example.

(Light Transmission Unit)

A light transmission unit serves as means for guiding the light beam generated by the light source to an emission unit, to be described below. The light transmission unit is formed by connecting a plurality of hollow waveguides using joints having encased mirrors, and an articulated arm configured such that light can propagate through the waveguides or a device that guides light propagating through space using optical elements such as mirrors and lenses, for example, maybe used as the light transmission unit.

(Emission Unit)

The emission unit emits the light beam guided by the light transmission unit onto an object such as a living organism. An emission intensity, a light intensity distribution, and a position of the light beam emitted onto the object can be adjusted to favorable levels using optical elements such as mirrors, lenses, and prisms.

(Acoustic Wave Reception Element)

An acoustic wave reception element serves as means for receiving a photoacoustic wave generated when the energy of the light pulse emitted by the emission unit is absorbed by an absorber on the object surface or in the object interior, and converting the received photoacoustic wave into an analog electric signal (a reception signal). An element that uses piezoelectricity, an element that uses optical resonance, or an element that uses capacitance variation may be employed as the acoustic wave reception element. The present invention is not limited to this configuration, however, and any element capable of receiving acoustic waves may be employed. The acoustic wave reception element may be formed by disposing a plurality of piezo elements or the like one-dimensionally, two-dimensionally, or three-dimensionally, for example. When an acoustic wave reception element formed by disposing a plurality of piezo elements or the like (any elements capable of receiving acoustic waves) in multiple dimensions in this manner is used, acoustic waves can be received in a plurality of positions simultaneously, with the result that a measurement time can be shortened. When a plurality of acoustic wave reception elements are disposed three-dimensionally in an acoustic wave reception unit, the acoustic wave reception elements may be disposed such that respective directions thereof in which a reception sensitivity is highest are oriented toward (concentrated on) a fixed area of the object interior. For example, the plurality of acoustic wave reception elements may be disposed to extend around a substantially hemispherical surface shape.

In this embodiment, the plurality of acoustic wave reception elements and supports for supporting the acoustic wave reception elements together constitute a conversion unit.

(Electric Signal Collection Unit)

An electric signal collection unit serves as means for collecting electric signals acquired by the acoustic wave reception elements. To ensure efficient processing, the electric signal collection unit preferably includes an A/D conversion unit that converts analog electric signals into digital signals.

(Holding Unit)

A holding unit serves as means used to hold the object, and may be a cup-shaped device that matches the shape of the object, a device constituted by two holding plates that sandwich the object fixedly, or any other device capable of holding the object, for example. When the holding unit is positioned between the object and the acoustic wave reception elements, the holding unit is preferably constituted by a device exhibiting poor light and acoustic wave absorption and having an acoustic impedance that is close to the acoustic impedance of the object. For example, the holding unit is preferably formed from a material such as polymethyl pentene resin or polyethylene terephthalate resin.

(Moving Unit)

A moving unit includes an XY stage 115 and a support table 113, to be described below, and serves as means for moving the emission unit in a two-dimensional direction. The moving unit may be provided with a position detection unit that detects the position of the emission unit during acoustic wave reception or light emission. The moving unit may be configured to move the acoustic wave reception elements and the emission unit integrally.

In this embodiment, the light source, the light transmission unit, the emission unit, and the moving unit together constitute a light emission unit.

(Drive Unit)

The drive unit serves as means for driving the moving unit on the basis of a drive command signal from a control unit. The drive unit performs this driving operation such that the moving unit moves the emission unit in a two-dimensional direction. The drive unit may be configured to drive the moving unit such that the emission unit moves continuously by uniform motion, or may be configured to drive the moving unit using a step and repeat method such that movement of the emission unit and acoustic wave reception are performed alternately. Further, the drive unit may be configured to drive the moving unit such that the emission unit moves in an arc shape or a spiral shape.

(Position Acquisition Unit)

A position acquisition unit serves as means for acquiring information indicating the position of the emission unit when the emission unit emits the light pulse onto the object. In a case where the acoustic wave reception elements and the emission unit are integrated, the position acquisition unit may be configured to acquire information indicating the positions of the acoustic wave reception elements when the light pulse is emitted onto the object at the same time as the information indicating the position of the emission unit is acquired. Note that when the position of the emission unit can be specified from the command signal issued to the drive unit by the control unit, the position acquisition unit may be omitted.

(Control Unit)

The control unit serves as means for controlling the entire apparatus so that the acoustic wave reception elements receive photoacoustic waves in desired positions and at desired timings. For this purpose, the control unit includes a light source control unit, a drive control unit, a collection control unit, and a system control unit (not shown), to be described below.

(Light Source Control Unit)

The light source control unit serves as means for controlling the light source so that the light pulse is generated at a desired timing. By controlling the light source in this manner, the light source control unit ensures that the light pulse can be emitted by the emission unit at a desired timing. For example, the light source control unit may control the light source such that the light pulse is generated at a predetermined repetition frequency, or may control the light source such that the light pulse is generated on the basis of the information indicating the position of the emission unit.

(Drive Control Unit)

The drive control unit outputs the drive command signal to the drive unit. The drive unit moves the emission unit in the manner described above on the basis of the drive command signal. Further, the drive control unit outputs a position acquisition command signal to the position acquisition unit. On the basis of the position acquisition command signal, the position acquisition unit acquires information indicating the position of the emission unit immediately after the emission unit emits the light pulse onto the object. The drive control unit may be provided with a separate function allowing an operator to specify an area of interest so that acoustic wave information relating to a specific area of the object can be acquired, and may issue a scanning command corresponding to this area of interest to the drive unit.

(Collection Control Unit)

The collection control unit outputs a collection command signal to the electric signal collection unit. The electric signal collection unit receives the collection command signal. On the basis of the received collection command signal, the electric signal collection unit acquires electric signals from the moment the light pulse is emitted onto the object to a time corresponding to a depth of the object at which an image is to be formed (an image is to be reconstructed). Alternatively, the electric signal collection unit obtains electric signals from a point following the elapse of a fixed time after the light pulse is emitted onto the object to the time corresponding to the depth of the object at which an image is to be formed (an image is to be reconstructed). The collection control unit controls a timing at which the electric signal collection unit acquires the analog electric signals output by the acoustic wave reception elements after receiving the photoacoustic waves, and a corresponding acquisition period.

(System Control Unit)

The system control unit controls the light source control unit, the drive control unit, and the collection control unit so that the photoacoustic waves can be received at desired timings.

(Distribution Storage Unit)

A distribution storage unit stores information relating to a light intensity profile (a two-dimensional spatial distribution) of a beam section of an emitted light beam immediately after the light beam is emitted from an emission end (corresponding to an emission end of an emission optical system) of the emission unit. In the present invention, the light intensity profile will also be referred to as a beam profile. The light intensity profile of the beam section may be the light intensity profile of the beam section immediately after the light beam is emitted from the emission unit, for example. However, the present invention is not limited to this configuration, and the light intensity profile of the beam section may also be the light intensity profile of the beam section between the emission end of the emission unit, from which the light beam is emitted, and the object. The distribution storage unit may store the information relating to the light intensity profile of the beam section when the emission unit is disposed in a predetermined light emission position (corresponding to an emission position). Alternatively, when a plurality of light emission positions exist, the distribution storage unit may store information relating to the light intensity profile of the beam section in each emission position. Note that here, the light beam may be a light beam such as a laser beam having a definable cross-section. The beam section may be a cross-section obtained when the light beam is cut on an orthogonal plane to an advancement direction of the light beam, or a cross-section obtained when the light beam is cut on a plane oriented in a diagonal direction relative to the advancement direction of the light beam, for example.

(Variation Storage Unit)

A variation storage unit stores variation information, which is information relating to variation in the light intensity profile of the beam section based on variation in the position of the emission unit. The variation storage unit may store the variation information relating to all of the light emission positions of the emission unit, or only the variation information relating to a plurality of representative light emission positions. The variation information includes information indicating either positional deviation or rotational deviation in a translational direction in the light intensity profile of the beam section, and may be information indicating variation in the emitted light beam either in the vicinity of the emission end of the emission unit or in a case where a hypothetical screen is disposed in a position removed from the emission end by a fixed distance. The variation storage unit may store the variation information in association with the light emission position of the emission unit.

(Interpolation Unit)

An interpolation unit serves as means for generating interpolated variation information by interpolating the variation information stored in the variation storage unit when the position of the emission unit associated with the variation information stored in the variation storage unit differs from the actual position in which the emission unit emits the light pulse onto the object.

(Variation Information Generation Unit)

A variation information generation unit serves as means for acquiring in advance a relational expression expressing a relationship between the position of the emission unit and the variation information, and generating the variation information in the position of the emission unit on the basis of the relational expression and information indicating the actual position in which the emission unit emits the light pulse onto the object. When the object information acquiring apparatus includes the variation information generation unit, the variation storage unit may be omitted. Note that a relationship table may be used instead of the relational expression. Further, a beam profile corresponding to the actual position in which the light pulse is emitted onto the object will also be referred to as a reference beam profile.

(Signal Processing Unit)

A signal processing unit serves as means (a characteristic information acquisition unit) for generating a three-dimensional photoacoustic wave image or an optical characteristic distribution of the object interior from the electric signals collected by the electric signal collection unit. The signal processing unit may generate the photoacoustic wave image using a UBP (Universal Back Projection) algorithm or a Delay and Sum algorithm, for example. The signal processing unit may also generate information indicating a three-dimensional light fluence distribution of the object interior on the basis of variation information. This variation information may be generated on the basis of at least one of the variation information stored in the variation storage unit, the interpolated variation information generated by the interpolation unit, and the variation information generated by the variation information generation unit, as well as the information relating to the light intensity profile of the beam section, which is stored in the distribution storage unit.

The signal processing unit may generate the information indicating the three-dimensional light fluence distribution of the object interior from the information relating to the two-dimensional light intensity profile of the beam section by solving an optical diffusion equation. The signal processing unit may acquire a light absorption coefficient distribution of the object interior by normalizing the photoacoustic wave image using the three-dimensional light fluence distribution information. Further, when light pulses of a plurality of wavelengths are emitted onto the object, the signal processing unit may perform image reconstruction at each of the wavelengths. In so doing, the signal processing unit can determine the light absorption coefficient distribution at each wavelength, and obtain an oxygen saturation distribution of hemoglobin in the object on the basis of the light absorption coefficient distribution at each wavelength.

### First Embodiment

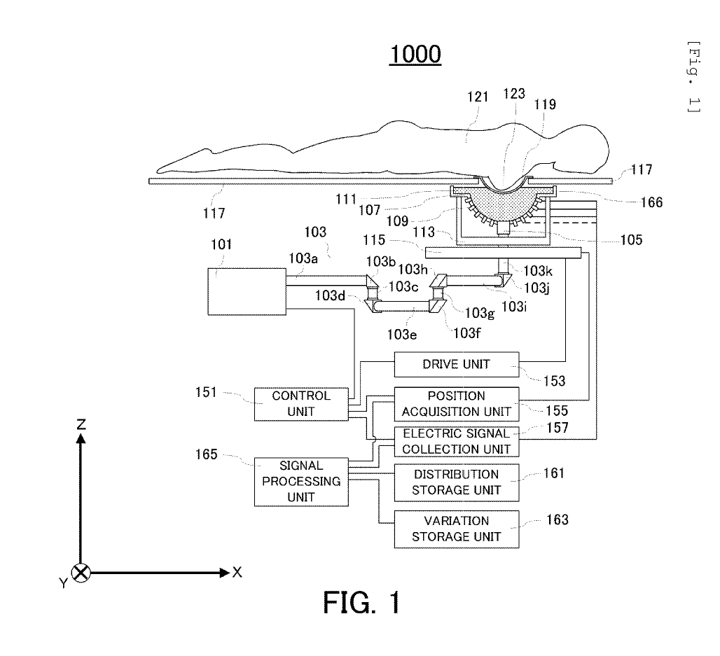

FIG. 1 is a block diagram showing a first embodiment of the object information acquiring apparatus according to the present invention. In an object information acquiring apparatus 1000 (abbreviated hereafter to “the apparatus 1000”) according to the first embodiment, abed 117, a light source 101, an articulated arm 103, an emission unit 105, an acoustic wave reception unit 166, a control unit 151, a signal processing unit 165, and so on are formed on a base.

The light source 101 may be formed from a titan-sapphire laser that generates a light pulse having a wavelength of 800 nm, a pulse width of 20 nsec, a repetition frequency of 10 Hz, and a pulse energy of 30 mJ. The articulated arm 103 is constituted by horizontal waveguides 103a, 103e, 103i, vertical waveguides 103c, 103g, 103k, and joints 103b, 103d, 103f, 103h, 103j encasing 45-degree mirrors. A propagation direction of a light beam propagating through the waveguides 103a, 103e, 103i, 103c, 103g, 103k is varied by 90 degrees at each joint 103b, 103d, 103f, 103h, 103j.

The horizontal waveguide 103a, the joint 103b, and the vertical waveguide 103c are connected fixedly so as to be incapable of moving. The joint 103d, the horizontal waveguide 103e, and the joint 103f are integrated and connected such that relative positional relationships with each other are fixed. The joint 103h, the horizontal waveguide 103i, and the joint 103j are integrated and connected such that relative positional relationships with each other are fixed. The joints 103d, 103f, 103h, and 103j are configured to be capable of rotating in a horizontal plane (in an XY plane) using the vertical waveguides 103c, 103g, 103k connected respectively thereto as rotary axes that are parallel to a Z axis. As a result, the vertical waveguide 103k is capable of parallel motion or rotation in a horizontal plane. Note that the present invention is not limited to this configuration, and depending on the requirements of the apparatus 1000, only a part of the joints 103d, 103f, 103h, and 103j may be configured to be rotatable.

The acoustic wave reception unit 166 may be configured such that a plurality of acoustic wave reception elements 109 and the emission unit 105 are supported by a substantially hemispherical surface-shaped support 107. The acoustic wave reception unit 166 may be formed integrally with the support 107 and the emission unit 105 so as to be capable of holding an acoustic matching agent 111.

The emission unit 105 encases a concave lens (not shown) for enlarging a light beam, and is configured to be connectable to a final end portion of the vertical waveguide 103k. Note that the present invention is not limited to this configuration, and the concave lens may be provided separately rather than being encased in the emission unit 105. In this embodiment, the articulated arm 103 and the emission unit 105 together form an emission optical system. The emission unit 105 may be considered as an emission end of the emission optical system.

The plurality of acoustic wave reception elements 109 are supported by the support 107 so as to extend around the substantially hemispherical surface shape thereof, and the directions of the respective acoustic wave reception elements 109 in which the reception sensitivity is highest are oriented toward a curvature center of the substantially hemispherical surface shape. As a result, a highly sensitive area in which photoacoustic waves can be received by the respective acoustic wave reception elements 109 with a high degree of sensitivity is formed in the curvature center of the substantially hemispherical surface shape of the support 107 and in the vicinity of the curvature center. The acoustic wave reception elements 109 may be transducers formed from piezoelectric elements that have a 3 mm-square element size and are capable of detecting acoustic waves with a center frequency of 2 MHz. 500 acoustic wave reception elements 109 may be arranged around the substantially hemispherical surface shape, and a radius of the substantially hemispherical surface shape may be set at 10 cm.

The support 107 serves as means for supporting the emission unit 105 and the acoustic wave reception elements 109. The support 107 is formed in the shape of a substantially hemispherical surface, and the acoustic wave reception elements 109 are supported thereby so as to extend around the substantially hemispherical surface. The support 107 may be formed integrally with the acoustic wave reception elements 109 and the emission unit 105 so as to be capable of holding the acoustic matching agent 111. Note that the emission unit 105 and the acoustic wave reception elements 109 may be formed separately to the support 107 as well as being supported by the support 107.

The support table 113 serves as means for supporting the support 107, and is configured to be capable of moving in an XY plane direction. The support table 113 is configured to be capable of moving the support 107 in the XY plane direction by moving in the same direction. The support table 113 is disposed on the XY stage 115, and can be moved in the XY plane direction by the XY stage 115. The XY stage 115 can be moved by a drive unit 153. The XY stage 115 further includes a position sensor (not shown). The position sensor detects the position of the emission unit 105, and transmits position information indicating the detection result to a position acquisition unit, to be described below. The position sensor may be configured to detect position information based on an X coordinate and a Y coordinate on the basis of an amount by which the XY stage 115 is driven. Note that the drive unit 153 may be a motor driver serving as a device for driving the XY stage. Further, the drive unit 153 may drive the XY stage on the basis of a command from the control unit 151.

The acoustic matching agent 111 is provided between a holding cup 119 and the acoustic wave reception elements 109 as a member for acoustically linking the holding cup 119 to the acoustic wave reception elements 109. The present invention is not limited to this configuration, however, and in a case where the holding cup 119 is not provided, the acoustic matching agent 111 may be provided so as to link an object 123 acoustically to the acoustic wave reception elements 109. Furthermore, the acoustic matching agent 111 may be any substance through which a photoacoustic wave generated from the object 123 can propagate efficiently. Water, oil, or the like, for example, is used as the acoustic matching agent 111.

The bed 117 is configured so that a person 121, for example, can lie face down thereon and insert the object 123, such as a breast, into an opening therein.

The holding cup 119 may be provided so as to be fitted into the opening in the bed 117. Ultrasound gel is provided between the holding cup 119 and the object 123 as an acoustic linker, and the holding cup 119 is acoustically linked to the object 123 by the ultrasound gel.

The light pulse generated by the light source 101 propagates through the articulated arm 103 so as to be emitted onto the object 123 via the emission unit 105, the acoustic matching agent 111, the holding cup 119, and the ultrasound gel. The acoustic matching agent 111, the holding cup 119, and the ultrasound gel are preferably configured so as to transmit the light pulse.

The control unit 151 includes a light source control unit, a drive control unit, a collection control unit, and a system control unit for controlling the other units (none of which are shown in the drawing). The control unit 151 is constituted by a calculation element such as a CPU, for example. The light source control unit controls the light source 101 to generate the light pulse at a desired timing. The light source control unit may control the light source 101 so that the light source 101 generates the light pulse at a repetition frequency of 10 Hz, for example. The drive control unit controls the drive unit 153 so that a desired movement is applied to the emission unit 105. For example, the drive control unit may control the drive unit 153 to move the emission unit 105 in a spiral shape. Further, the drive control unit issues a command to the position acquisition unit 155 to acquire from the aforesaid position sensor information indicating the position of the emission unit 105 at the moment when the light pulse is emitted onto the object 123. In this embodiment, the emission unit 105 is integrated with the acoustic wave reception elements 109 by the support 107, and therefore the acquired information indicating the position of the emission unit 105 doubles as information indicating positions in which the acoustic wave reception elements 109 acquire electric signals. Note that the position acquisition unit 155 may acquire the position of the XY stage. For example, the XY stage may be provided with an encoder so that the position acquisition unit 155 can acquire information indicating the position of the XY stage on the basis of information relating to the encoder. Moreover, the present invention is not limited to this configuration, and the functions of the position acquisition unit 155 maybe included in the drive unit 153.

With this configuration, labor required to acquire the information indicating the positions in which the acoustic wave reception elements 109 acquire electric signals separately from the information indicating the position of the emission unit can be eliminated, and as a result, the time expended by the apparatus 1000 on signal processing when acquiring the object information can be shortened. The present invention is not limited to this configuration, however, and the information indicating the positions in which the acoustic wave reception elements 109 acquire electric signals may be determined by calculation on the basis of the emission position of the emission unit. The collection control unit issues a command to an electric signal collection unit 157 to collect signals reaching the acoustic wave reception elements 109 over a period extending from a time 50 μsec (microseconds) to a time 100 μsec, where a time at which the light pulse is emitted onto the object 123 is a time 0 μsec. Signals reaching the acoustic wave reception elements 109 between the time 0 μsec and the time 50 μsec are signals generated from the acoustic matching agent 111, and are therefore meaningless as data. Hence, these signals are not collected. The signals reaching the acoustic wave reception elements 109 between the time 50 μsec and the time 100 μsec include signals from the object 123, and are therefore collected.

Note that the control unit 151 may be a CPU including a control program. The control unit 151 may be configured to operate an operating system (OS) that performs basic resource control, management, and so on during a program operation.

Further, the electric signal collection unit 157 may be configured to amplify the electric signals generated by the respective acoustic wave reception elements 109 either separately or all together and then convert the amplified signals into digital signal data. The electric signal collection unit 157 may be formed from a signal amplification unit (an operational amplifier or the like) that amplifies the generated analog signals, and an A/D conversion unit that converts the analog signals into digital signals. When the amount of data is large, the electric signal collection unit 157 may be formed from a dedicated IC (also referred to as a Data Acquisition System) such as an FPGA.

The distribution storage unit 161 stores the information relating to the light intensity profile of the beam section. The information relating to the light intensity profile of the beam section may be information configured as follows. Specifically, when the emission unit 105 is placed in the center of a range in which a light beam can be emitted and an acoustic wave based on the emitted light beam can be acquired, and a screen is disposed in a position 10 cm from the emission unit 105, the light intensity profile of a cross-section formed by a light beam emitted onto the screen may be measured and acquired as the information relating to the light intensity profile of the beam section. The center of the range in which an acoustic wave can be acquired is directly below a deepest portion of the holding cup 119, for example. Note that the light intensity profile of the beam section of the light beam emitted from the light source 101 varies as the light beam passes through the emission optical system. Hence, the distribution storage unit 161 may store the light intensity profile of the beam section at the point where the light beam is emitted after passing through the emission optical system in relation to each light emission position. The light intensity profile of the beam section at the point where the light beam is emitted from the emission end of the emission optical system after passing through the emission optical system may be the light intensity profile of the beam section as formed when the variation described above is taken into account.

The variation storage unit 163 stores information indicating variation in the light intensity profile of the beam section in each light emission position of the emission unit 105 as the variation information described above.

Storage means such as the distribution storage unit 161 and the variation storage unit 163 may be formed from a non-temporary storage medium such as a ROM (Read Only Memory), a magnetic disk, or a flash memory. Alternatively, the storage means may be a volatile medium such as a RAM (Random Access Memory). Note that a non-temporary storage medium is used as a storage medium for storing a program.

The signal processing unit 165 first generates a three-dimensional photoacoustic wave image of the interior of the object 123 by implementing signal processing using a UBP algorithm on the electric signals collected by the electric signal collection unit 157 in the respective light emission positions of the emission unit 105. The light emission positions of the emission unit 105 may also serve as electric signal acquisition positions. Further, the signal processing unit 165 generates information indicating a three-dimensional light fluence distribution (a light fluence distribution) of the interior of the object 123 using an optical diffusion equation on the basis of the information relating to the light intensity profile of the beam section, stored in the distribution storage unit 161, and the variation information stored in the variation storage unit 163, this information having been acquired in relation to each light emission position of the emission unit 105. Furthermore, the signal processing unit 165 acquires a light absorption coefficient distribution of the interior of the object 123 in relation to each light emission position of the emission unit 105 by normalizing the photoacoustic wave image using the three-dimensional light fluence distribution information. The signal processing unit 165 implements the respective processes described above in all of the light emission positions of the emission unit 105. When the processing described above is complete, the signal processing unit 165 superimposes the acquired light absorption coefficient distribution data on the acquired photoacoustic wave image data, thereby acquiring three-dimensional photoacoustic wave image data and light absorption coefficient distribution data for the entire object 123.

Note that since an information processing amount is large, the signal processing unit 165 preferably has a high-performance calculation processing function. Moreover, for the same reason, the signal processing unit 165 is preferably constituted by a multicore CPU or the like. Further, a processor such as a CPU, a GPU (Graphics Processing Unit), or a DSP (Digital Signal Processor) and an arithmetic circuit such as an FPGA (Field Programmable Gate Array) chip may serve as units for realizing the calculation functions of the signal processing unit 165. Either a single processor and a single arithmetic circuit or a plurality of processors and arithmetic circuits may be provided as these units.

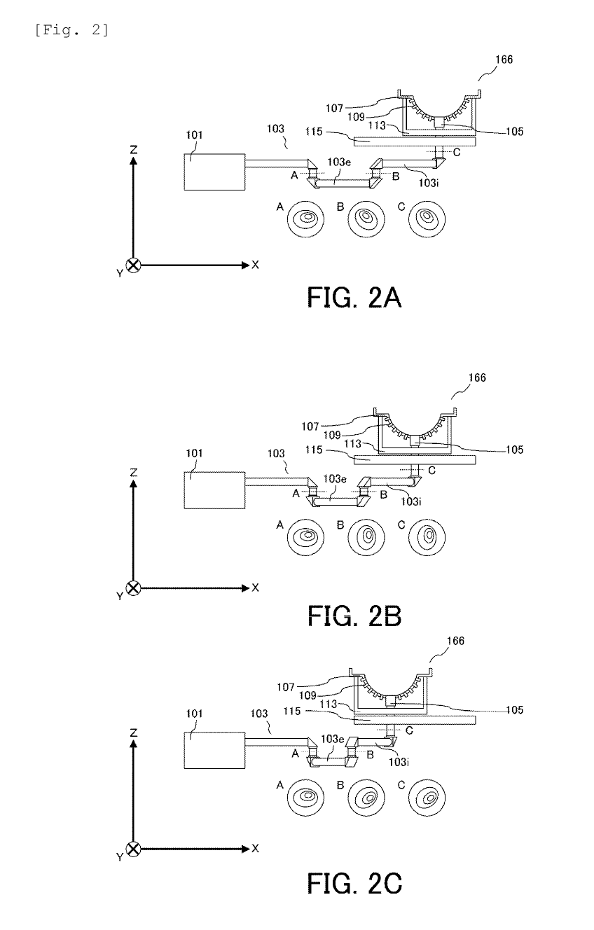

FIGS. 2A to 2C are pattern diagrams showing rotation of the light intensity profile of the beam section, according to the first embodiment. Parts corresponding to FIG. 1 have been allocated identical reference numerals, and description thereof has been omitted when not required. Note that parts above the bed 117 are not shown in FIGS. 2A to 2C

FIG. 2A shows a condition in which the emission unit 105 has been moved in a +X direction from a predetermined central position (a rotation center of a spiral movement performed by the emission unit 105, for example). At this time, elbows of the articulated arm 103 are said to be in an extended condition. FIG. 2A envisages a case in which, for example, hypothetical screens (indicated by A, B, and C in the drawing) are disposed in the three vertical waveguides of the articulated arm 103, and the light intensity profiles of the beam sections of light beams emitted thereon are observed from the bed side. The light intensity profiles of the beam sections of the light beams emitted onto the screens A, B, C are shown respectively in circles in the drawing. The light intensity profile of the beam section of the light beam emitted onto the screen A has a mirror image relationship with the light intensity profile of the beam section of the light beam emitted onto the screen B when a cross-section of the horizontal waveguide 103e is used as a plane of symmetry. Therefore, the light intensity profile of the beam section of the light beam emitted onto the screen B differs from the light intensity profile of the beam section of the light beam emitted onto the screen A in that the light intensity profile of the beam section is reversed about the cross-section of the horizontal waveguide 103e and rotated in accordance with the angles of the elbows. Further, the shape of the light intensity profile of the beam section of the light beam emitted onto the screen B is maintained (not rotated) even when the light beam is guided through the horizontal waveguide 103i, and therefore the shape of the light intensity profile of the beam section of the light beam emitted onto the screen C does not vary.

FIG. 2B shows a condition in which the emission unit 105 has been moved in a −X direction from the condition shown in FIG. 2A. In this case, the elbows of the articulated arm 103 are bent by 45 degrees. Likewise in FIG. 2B, similarly to FIG. 2A, the light intensity profiles of the beam sections of the light beams emitted onto the screens A, B, C are shown respectively in circles in the drawing.

FIG. 2C shows a condition in which the emission unit 105 has been moved further in the −X direction from the condition shown in FIG. 2B. In this case, the elbows of the articulated arm 103 are bent by an angle exceeding 45 degrees. Likewise in FIG. 2C, similarly to FIG. 2A, the light intensity profiles of the beam sections of the light beams emitted onto the screens A, B, C are shown respectively in circles in the drawing. As shown in FIGS. 2A to 2C, the light intensity profile of the beam section of the light beam emitted onto the screen C (the final light intensity profile of the beam section) rotates gradually clockwise as the elbows of the articulated arm 103 are bent.

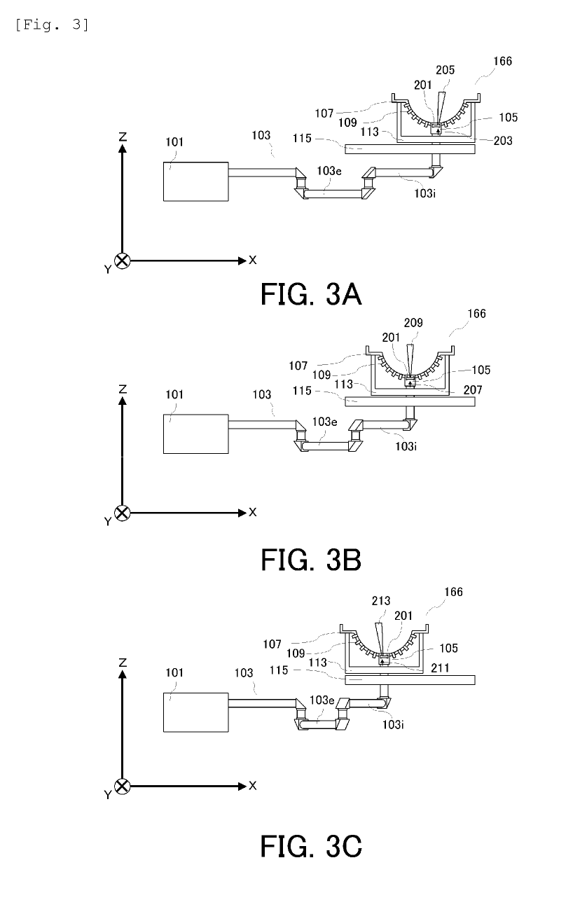

FIGS. 3A to 3C are pattern diagrams showing positional deviation in the light intensity profile of the beam section, according to the first embodiment. Parts corresponding to FIG. 1 have been allocated identical reference numerals, and description thereof has been omitted when not required. FIGS. 3A to 3C illustrates positional deviation among the light intensity profiles of the beam sections of light beams emitted in three light emission positions. Note that parts above the bed 117 are not shown in FIGS. 3A to 3C.

FIG. 3A shows a condition in which the emission unit 105 has been moved in the +X direction from a predetermined central position (the rotation center of the spiral movement performed by the emission unit 105, for example). The emission unit 105 includes a concave lens 201 for spreading the light (the light beam) from the light source 101. The concave lens 201 is provided so that the light from the light source 101 can be spread (dispersed) to a certain extent before being emitted onto the object 123. In so doing, a photoacoustic wave can be generated from the absorber more efficiently, with the result that the object information can be acquired more efficiently. The concave lens 201 may be provided so as to be encased in the emission unit 105, or provided separately on the outside of the emission unit 105. In FIG. 3A, the light beam from the light source 101 enters the concave lens 201 from the right side. Therefore, the light advances with a center of gravity 203 thereof refracted rightward in accordance with the curvature of the concave lens 201. Note that here, a center of gravity g of the light beam maybe defined as a point g satisfying Expression (1), where dS is an area element, when each point r on a two-dimensional graphic S formed from a light intensity distribution of the cross-section of the light beam has a light intensity density f(r), for example.

∫ₛ (g−r) f (r) ds=0 Expression (1)

FIG. 3B shows a condition in which the emission unit 105 has been moved in the −X direction from the condition shown in FIG. 3A. FIG. 3B shows a case in which a center of gravity 207 of the light beam enters the center of the concave lens 201, with the result that the center of gravity 207 of the incident light beam is not refracted. Accordingly, a light beam 209 emitted from the concave lens 201 is enlarged by the concave lens 201 as is, i.e. without being refracted.

FIG. 3C shows a condition in which the emission unit 105 has been moved further in the −X direction from the condition shown in FIG. 3B. In FIG. 3C, the light beam from the light source 101 enters the left side of the concave lens 201. The concave lens 201 disperses the light beam entering the left side, and therefore a center of gravity 211 of the light beam is refracted leftward. As a result, a light beam 213 emitted from the emission unit 105 is refracted leftward, spread, and emitted thus onto the object 123.

As illustrated in FIGS. 2A to 2C, the center of gravity of the light beam entering the concave lens 201 is moved rotationally by bending the elbows of the articulated arm 103. Hence, the center of gravity of the light beam entering the concave lens 201 rotates in accordance with the extent to which the elbows of the articulated arm 103 are bent. Accordingly, the position of the center of gravity of the light beam entering the concave lens 201 may deviate from the center position of the concave lens 201 by rotating in accordance with the extent to which the elbows of the articulated arm 103 are bent. When the position of the center of gravity of the light beam entering the concave lens 201 deviates from the center position of the concave lens 201, the light intensity profile of the beam section of the light beam emitted from the emission unit 105 rotates together with the center of gravity of the light beam. Therefore, by bending the elbows of the articulated arm 103 sequentially as shown in FIGS. 3A, 3B, and 3C, the direction in which the light beam advances after passing through the concave lens 201 shifts from a rightward direction to a leftward direction. Note that for ease of description, this movement is expressed two-dimensionally, but in actuality, the movement occurs in three dimensions.

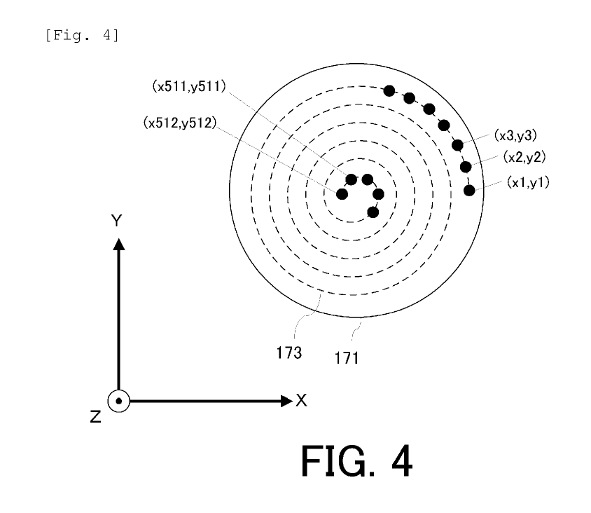

FIG. 4 is a pattern diagram showing the light emission positions of the emission unit 105 according to the first embodiment. FIG. 4 shows a scannable range 171 and a movement locus 173 of the emission unit 105. Black circles in FIG. 4 indicate respective positions of the emission unit 105 at the moments when light pulses are emitted onto the object 123. In this embodiment, a screen is disposed in a position located 10 cm away from the emission unit 105 toward the object 123 side. The light intensity profiles of the beam sections of light beams formed on the screen when light is emitted by the emission unit 105 in 512 positions (the positions of the black circles) are measured in advance. The variation information in each light emission position (the position of each black circle) is then calculated on the basis of the measurement results.

The acoustic wave reception unit 166 may be controlled such that light pulses are emitted by the emission unit 105 integrated therewith 512 times at a repetition frequency of 10 Hz while the emission unit 105 moves in a spiral shape, for example. In this case, the emission unit 105 is positioned in a total of 512 locations at the moments when the light pulses are emitted onto the object 123.

The variation storage unit 163 stores the variation information calculated in this manner.

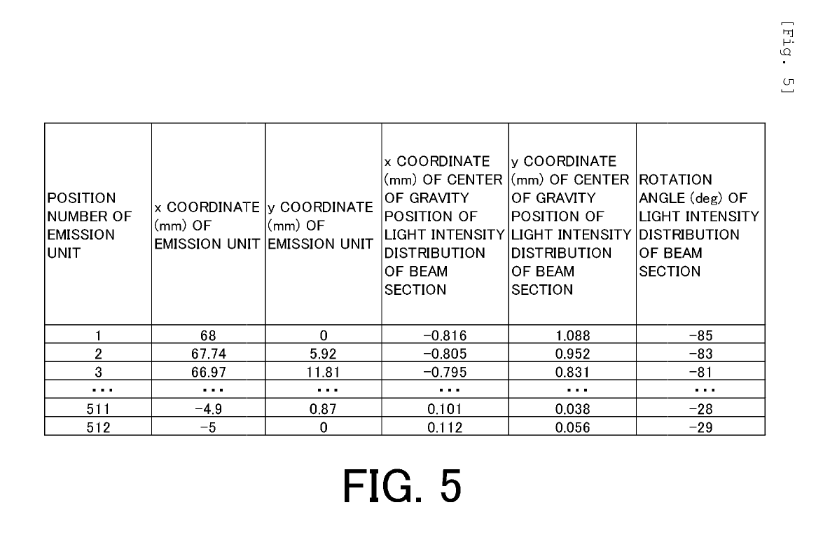

FIG. 5 is a table showing the variation information stored in the variation storage unit 163 according to the first embodiment. In FIG. 5, first, second, third, fourth, fifth, and sixth columns from the left show a position number of the emission unit, an x coordinate and a y coordinate (mm) of the emission unit, an x coordinate and a y coordinate (mm) of a center of gravity position of the light intensity profile of the beam section, and a rotation angle (deg) of the light intensity profile of the beam section, respectively. Information may be stored in the variation storage unit 163 such that these respective elements are associated with each other. The present invention is not limited to this configuration, however, and various other storage methods may be applied to the variation storage unit 163.

In this embodiment, as described above, information indicating the three-dimensional light fluence distribution of the interior of the object 123 is generated using an optical diffusion equation on the basis of the information relating to the light intensity profiles of the beam sections, stored in the distribution storage unit 161, and the variation information stored in the variation storage unit 163. In so doing, the three-dimensional light fluence distribution of the interior of the object 123 in each light emission position of the emission unit 105 can be generated more accurately.

Furthermore, by normalizing the photoacoustic wave images acquired in the same light emission positions using the three-dimensional light fluence distribution information, the light absorption coefficient distribution of the interior of the object 123 in each light emission position of the emission unit 105 can be acquired more accurately.

By implementing these processes over an entire area to be turned into an image and finally superimposing the acquired light absorption coefficient distributions, the three-dimensional light absorption coefficient distribution of the entire object 123 can be acquired with a high degree of precision. Note that the present invention is not limited to the calculation sequence described above, and instead, a three-dimensional photoacoustic wave image of the entire object 123 may be acquired first. The information indicating three-dimensional light fluence distribution of the entire object 123 may then be acquired, whereupon the three-dimensional light absorption coefficient distribution of the entire object 123 may be determined.

Note that the holding cup 119 may be configured to be capable of aligning the shape of the object 123 with the shape of the holding cup 119 (a cup shape, for example) by holding the object 123. Accordingly, the apparatus 1000 may calculate the three-dimensional light fluence distribution of the interior of the object 123, formed when the emission unit 105 emits light beams in the light emission positions described above, on the basis of a light intensity profile formed on the surface of the holding cup 119 when the light pulse impinges on the surface of the holding cup.

The shape of the holding cup 119 is known. Therefore, the light intensity profile formed on the surface of the holding cup in each light emission positon when the light pulse impinges on the surface of the holding cup 119 may be calculated from the light emission position of the emission unit 105, the light intensity profile of the beam section in the corresponding light emission position, a magnification of the concave lens 201, and the known shape of the holding cup. The magnification of the concave lens 201 may be determined in advance by measurement or the like. The light intensity profile of the beam section in the light emission position and the light emission position of the emission unit 105 are already known, as described above, and therefore the light intensity profile on the surface of the holding cup 119 is likewise known from the above. Hence, the apparatus 1000 may determine the three-dimensional light fluence distribution information by performing a calculation on the basis of the light intensity profile on the surface of the holding cup 119. Note that the present invention is not limited to this configuration, and various other forms capable of maintaining the object 123 in a predetermined shape may be applied as the holding unit instead of the holding cup 119.

As a result, a calculation load required to calculate the three-dimensional light fluence distribution information can be reduced, and the time required to acquire the characteristic information of the object can be shortened.

Furthermore, the apparatus 1000 may store the three-dimensional light fluence distribution information determined in this manner in a memory or the like in advance. Then, when the three-dimensional light fluence distribution information is determined subsequently in the apparatus 1000, the three-dimensional light fluence distribution information stored in the memory can be read and used to acquire the object information, enabling a further reduction in the time required to acquire the characteristic information of the object.

Note that when the three-dimensional light fluence distribution information to be stored in the memory in advance is determined, an existing phantom, for example, may be used instead of the object 123. Further, instead of using a phantom, the three-dimensional light fluence distribution information to be stored in the memory in advance may be determined by assuming that the holding cup 119 is filled with fat, for example. In so doing, the three-dimensional light fluence distribution information to be stored in the memory in advance can be acquired easily. This applies similarly to other embodiments having a holding unit (the holding cup 119, for example), to be described below.

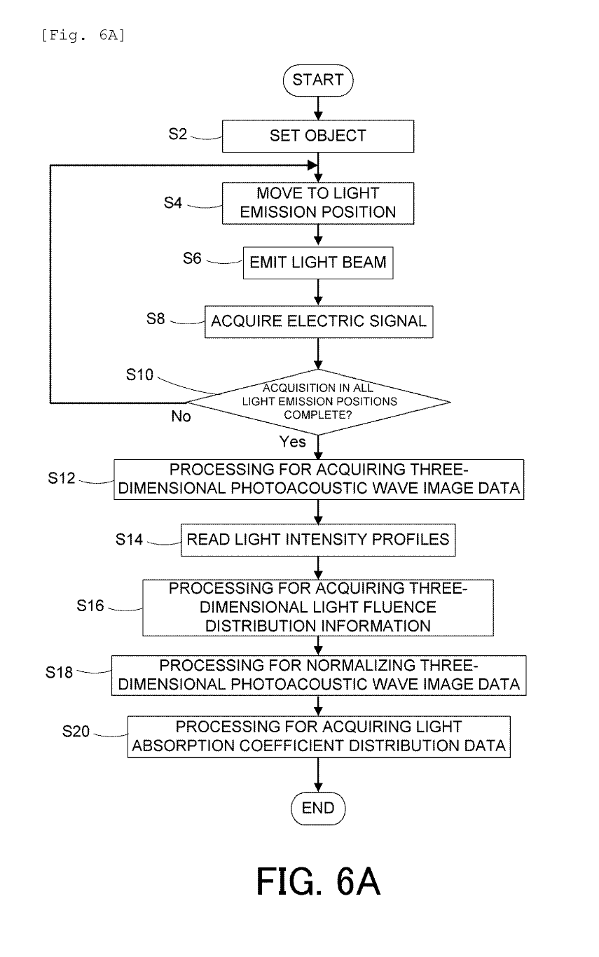

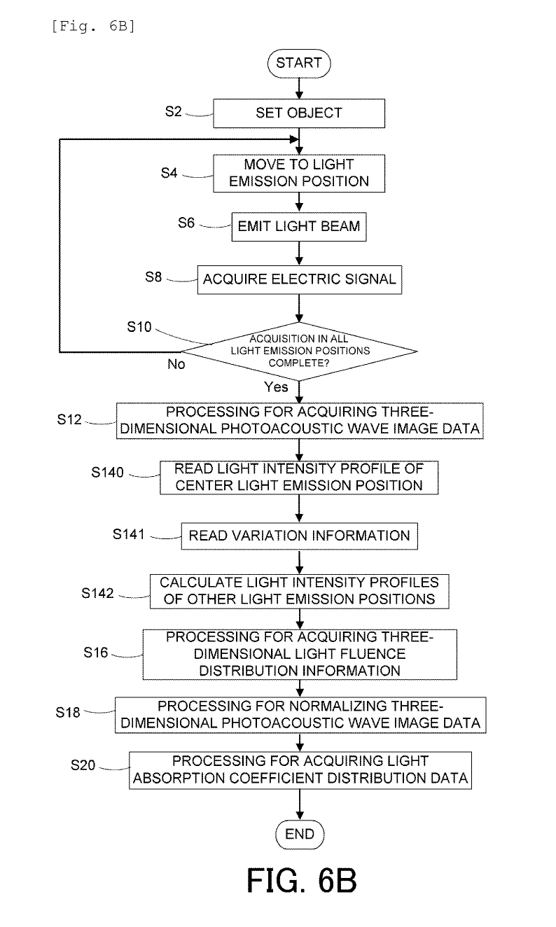

FIG. 6A is a flowchart showing an example of the functions of the apparatus 1000 according to the first embodiment. The flow is started when power is supplied to the apparatus 1000. In step S2, the object 123 is inserted into the opening provided in the bed 117. The object 123 is then set in the apparatus 1000 by being held by the holding cup 119, whereupon the flow advances to step S4. In step S4, the emission unit 105 is moved to the position in which a light beam is to be emitted onto the object 123, whereupon the flow advances to step S6. In step S6, a light beam is emitted by the emission unit 105 in the position to which the emission unit 105 has been moved, whereupon the flow advances to step S8. In step S8, an acoustic wave propagating through the object 123 in response to emission of the light beam is received by the acoustic wave reception unit 166 and converted into an electric signal that is acquired by the electric signal collection unit 157, whereupon the flow advances to step S10. In step S10, a determination is made as to whether or not electric signals have been acquired in all of the predetermined light emission positions. When it is determined that electric signals have been acquired in all of the 512 light emission positions, the flow advances to step S12, and when it is determined that electric signals have not been acquired in all of the light emission positions, the flow returns to step S4. In step S10, the processing of step S4 to step S8 is executed repeatedly until it is determined that electric signals have been acquired in all of the 512 light emission positions.

In step S12, image reconstruction is performed by the signal processing unit 165 on the basis of the acquired electric signals, whereby three-dimensional photoacoustic wave image data are acquired. In this case, three-dimensional voxel data are formed in each light emission position by performing image reconstruction processing using a UBP algorithm. The three-dimensional voxel data are then linked. Once three-dimensional photoacoustic wave image data including the entire object 123 have been acquired, the flow advances to step S14. In step S14, the information relating to the light intensity profile of the beam section in each light emission position is read from the distribution storage unit 161, whereupon the flow advances to step S16. In step S16, information indicating the three-dimensional light fluence distribution of the interior of the object 123 is acquired by the signal processing unit 165 in each light emission position on the basis of the read information relating to the light intensity profiles of the beam sections using an optical diffusion equation. The flow then advances to step S18. In step S18, the signal processing unit 165 normalizes (corrects) the three-dimensional photoacoustic wave image data on the basis of the acquired three-dimensional light fluence distribution information, thereby acquiring normalized three-dimensional photoacoustic wave image data. The flow then advances to step S20. In step S20, the three-dimensional light absorption coefficient distribution is superimposed on the normalized three-dimensional photoacoustic wave image data by the signal processing unit, whereby three-dimensional light absorption coefficient distribution data are acquired in relation to the object 123. The flow is then terminated. Note that in this case, the variation storage unit 163 does not have to be used.

FIG. 6B is a flowchart showing another example of the functions of the apparatus 1000 according to the first embodiment. FIG. 6B differs from FIG. 6A as follows. Firstly, the light intensity profile of the beam section is stored in the distribution storage unit 161 in relation to only one central position among the light emission positions. Secondly, information indicating a deviation from the light intensity profile of the beam section in this central position is read as appropriate from the variation storage unit 163. Thirdly, the light intensity profiles of the beam sections in the other light emission positions are acquired by applying the read deviation information to the light intensity profile of the beam section in the central position. The processing described above corresponds to steps S140 to S142. All other processing is identical to that of the flow shown in FIG. 6A, and will not therefore be described.

More specifically, when the processing from step S2 to step S12, which is similar to the processing of FIG. 6A, is complete, the flow advances to step S140. In step S140, the light intensity profile of the beam section in the central light emission position is read from the distribution storage unit 161, whereupon the flow advances to step S141. In step S141, the variation information corresponding to the light emission positions is read from the variation storage unit 163 in relation to each light emission position, whereupon the flow advances to step S142. In step S142, the light intensity profiles of the beam sections in the light emission positions other than the central position are calculated by the signal processing unit 165 in relation to each light emission position on the basis of the light intensity profile of the beam section in the central light emission position and the variation information corresponding to the light emission positions other than the central position. Once this calculation processing has been executed in relation to all of the light emission positions, the flow advances to step S16. The processing from step S16 to step S20 is then executed in a similar manner to the processing of FIG. 6A, whereupon the flow is terminated.

Note that the present invention is not limited to this configuration, and the distribution storage unit 161 may store the deviation from two locations, for example, rather than the deviation from the single central light emission position. Further, the distribution storage unit 161 may store information indicating the deviation from the light intensity profile of the beam section in a light emission position other than the central position.

### Second Embodiment

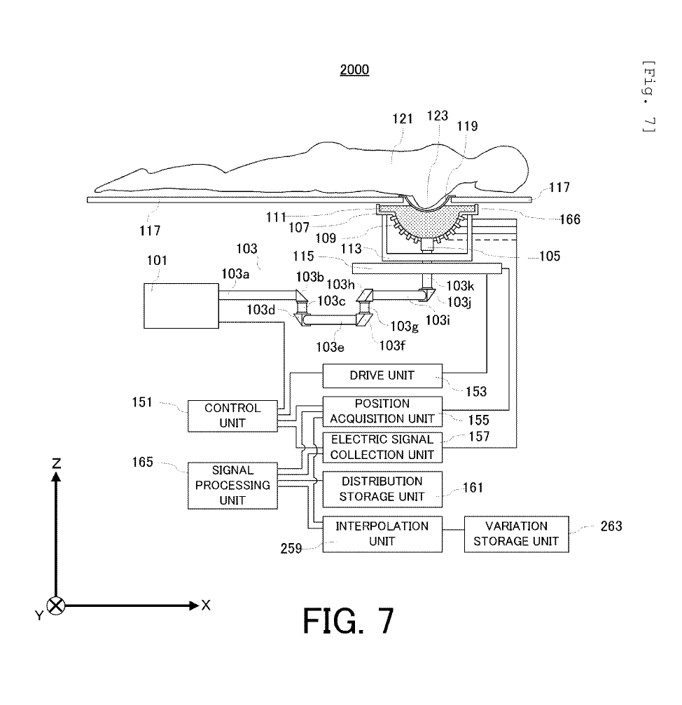

FIG. 7 is a block diagram showing a second embodiment of the object information acquiring apparatus according to the present invention. Configurations shared with the first embodiment have been allocated identical reference numerals, and description thereof has been omitted. An object information acquiring apparatus 2000 (abbreviated hereafter to “the apparatus 2000”) according to this embodiment stores only variation information relating to a plurality of representative light emission positions rather than storing the variation information relating to all of the light emission positions in which light beams are emitted onto the object 123. The apparatus 2000 generates the variation information in the actual light emission positions through interpolation. The apparatus 2000 differs from the apparatus 1000 according to the first embodiment on this point. To realize this configuration, the apparatus 2000 includes a variation storage unit 263 and an interpolation unit 259.

The variation storage unit 263 stores variation information in relation to 17 representative light emission positions.

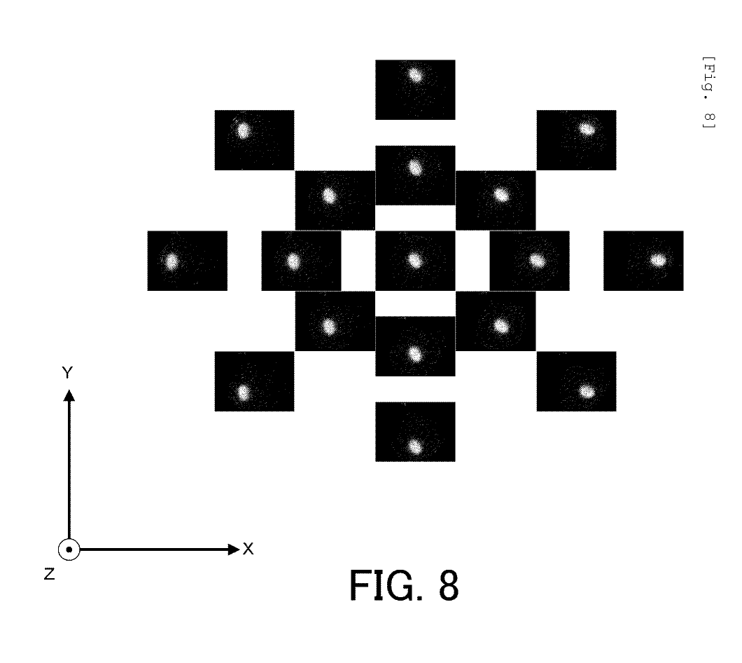

FIG. 8 is a view showing variation in the light intensity profile of the beam section according to the second embodiment. FIG. 8 shows actually measured variation in the light intensity profile of the beam section, and illustrates light intensity profiles of beam sections in a case where the emission unit 105 emits light in the following positions (X coordinate, Y coordinate): (−68, 0); (−34, 0); (0, 0); (34, 0); (68, 0); (0, −68); (0, −34); (0, 34); (0, 68); (−48, −48); (−24, −24); (24, 24); (48, 48); (−48, 48); (−24, 24); (−24, 24); (−48, 48) (unit: mm). As is evident from FIG. 8, the shape of the light intensity profile of the beam section rotates in accordance with the coordinates. It can be seen that the light intensity profile of the beam section rotates in accordance with the degree to which the elbows of the articulated arm 103 are bent. In FIG. 8, a drawing furthest toward the +X side represents the light intensity profile of the beam section when the elbows are fully extended, while a drawing furthest toward the −X side represents the light intensity profile of the beam section when the elbow on the left end is fully bent. In FIG. 8, the light intensity profile of the beam section rotates clockwise from the +X side toward the −X side, or in other words as the elbows of the articulated arm 103 bend. This is the result of the process illustrated in FIGS. 2A to 2C.

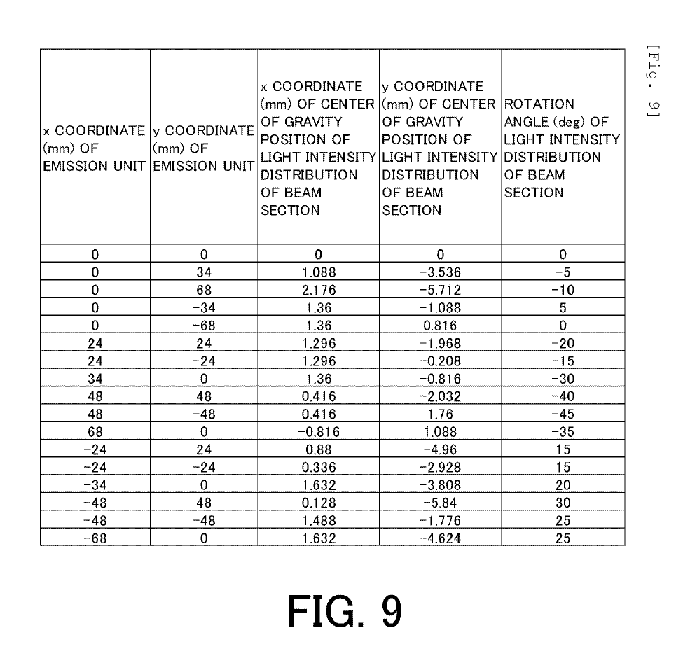

FIG. 9 is a table showing calculation results indicating amounts of variation in an emitted light beam, according to the second embodiment. The table shows, in order from the left-hand column, the x coordinate (mm) of the emission unit 105, the y coordinate (mm) of the emission unit 105, the x coordinate of the center of gravity position of the light intensity profile of the beam section, the y coordinate (mm) of the center of gravity position of the light intensity profile of the beam section, and the rotation angle (deg) of the light intensity profile of the beam section.

The variation storage unit 263 stores the table shown in FIG. 9 as the variation information.

Similarly to the first embodiment, the acoustic wave reception unit 166 may be controlled such that light pulses are emitted by the emission unit 105 integrated therewith 512 times at a repetition frequency of 10 Hz while the emission unit 105 moves in a spiral shape. In other words, the emission unit 105 may be positioned in a total of 512 locations at the moments when the light pulses are actually emitted onto the object 123.

The interpolation unit 259 may generate the variation information in each of the 512 actual light emission positions from the variation information stored in the variation storage unit 263 in relation to the 17 representative light emission positions shown in FIG. 9 through interpolation or extrapolation. The present invention is not limited to this configuration, however, and various methods, such as polynomial approximation, may be applied as the method used to generate the variation information in each of the 512 actual light emission positions. Note that the term interpolation is used to express data generation through interpolation or extrapolation. The amount of information processed by the interpolation unit 259 is smaller than that of the signal processing unit 165, and therefore the signal processing unit 165 may be configured to include the functions of the interpolation unit 259. In this case, the interpolation unit 259 maybe formed from the multicore CPU included in the signal processing unit 165.

A similar method to the first embodiment may be employed to acquire the photoacoustic wave image serving as a part of the object information. In this embodiment, only the variation information relating to the 17 representative light emission positions need be stored in advance in the variation storage unit 263, and therefore the object information can be acquired easily.

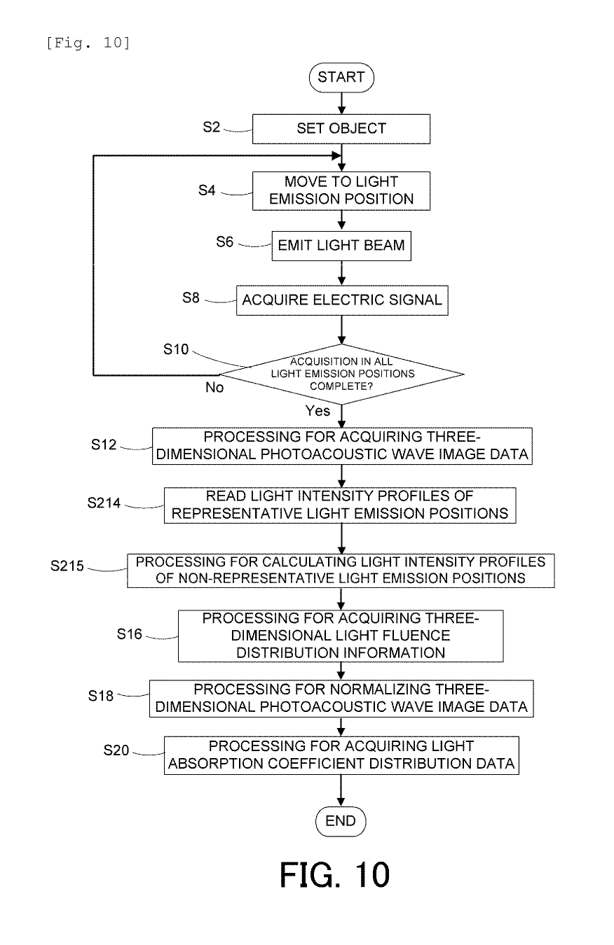

FIG. 10 is a flowchart showing functions of the apparatus 2000 according to the second embodiment. The processing from step S2 to step S12 and from step S16 to step S20 is identical to the processing of the flow shown in FIG. 6A or FIG. 6B, and therefore this processing will not be described. In other words, when the processing of step S2 to step S12, which is similar to the processing of FIG. 6A, is complete, the flow advances to step S214. In step S214, the light intensity profiles of the beam sections in the 17 representative light emission positions are read from the distribution storage unit 161, whereupon the flow advances to step S215.

In step S215, the light intensity profiles of the beam sections in the 512 light emission positions in which light beams are actually emitted are calculated by the interpolation unit 259. The 17 representative light emission positions may be selected from the 512 light emission positions in which light beams are actually emitted. In this case, the light intensity profiles of the beam sections in the 495 remaining light emission positions are calculated by the interpolation unit 259. After acquiring the light intensity profiles of the beam sections in the light emission positions in this manner, the flow advances to step S16, whereupon similar processing to that of FIGS. 6A and 6B is performed. Note that in this case, the variation storage unit 263 does not have to be used.

As another example, the light intensity profile of the beam section may be stored in the distribution storage unit 161 in relation to only one central position among the light emission positions. In this case, the light intensity profile of the beam section in the central light emission position may be read in step S214, whereupon the flow advances to step S215. In step S215, the interpolation unit 259 reads information indicating the deviation from the light intensity profile of the beam section in the central light emission position as appropriate from the variation storage unit 163, and generates deviation information for all of the 512 light emission positions. The light intensity profiles of the beam sections in all of the 512 light emission positions may then be acquired by applying the generated deviation information to the light intensity profile of the beam section in the central light emission position. The flow then advances as described above.

### Third Embodiment

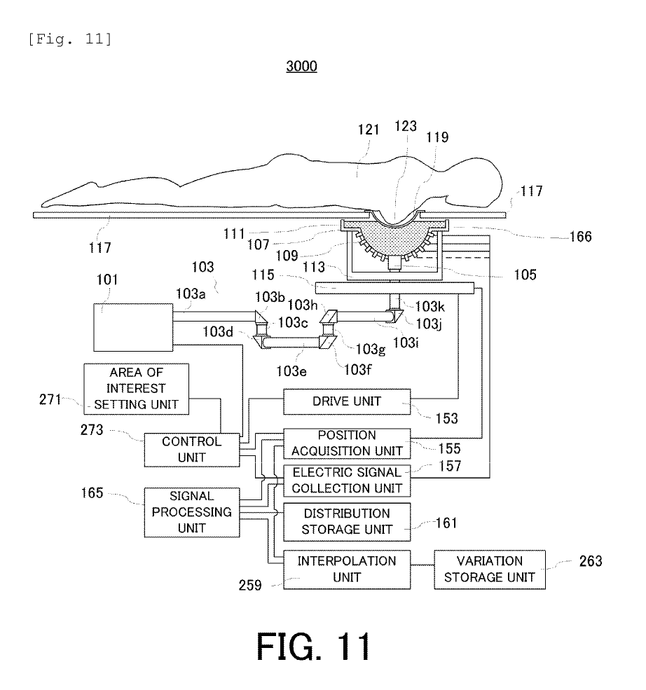

FIG. 11 is a block diagram showing a third embodiment of the object information acquiring apparatus according to the present invention. Parts corresponding to FIG. 1 or FIG. 7 have been allocated identical reference numerals, and description thereof has been omitted when not required. In an object information acquiring apparatus 3000 (abbreviated hereafter to “the apparatus 3000”) according to this embodiment, a case in which an area of interest is known in advance from palpation, an image generated using another imaging apparatus, such as an ultrasonic echo apparatus or an MRI apparatus, or the like is envisaged. The apparatus 3000 differs from the apparatus 200 according to the second embodiment in that an area of interest setting unit is provided. The apparatus 3000 includes an area of interest setting unit 271 and a control unit 273.

The area of interest setting unit 271 serves as means for setting an area of interest, which is an area of an object 315 (also including the periphery thereof) in which image reconstruction is to be performed. The apparatus 3000 includes a monitor (not shown), and the monitor is configured such that an operator can specify the area of interest thereon. The area of interest is set by the area of interest setting unit 271 on the basis of a specification result from the monitor.

The control unit 273 includes a drive control unit. The drive control unit may determine a movement range of the emission unit 105 as well as movement and light emission methods on the basis of the area of interest set by the area of interest setting unit 271. The drive control unit also inputs a signal serving as a determination result into the drive unit 153. The drive unit 153 drives the emission unit 105 in accordance with the determined movement range, movement method, and light emission method on the basis of the signal serving as the input result.

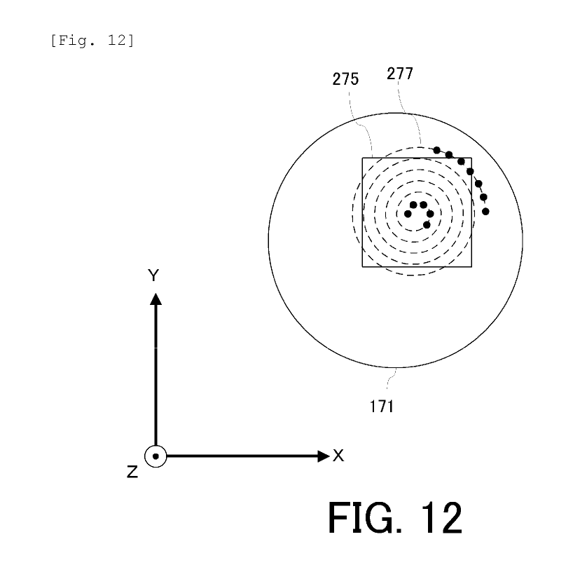

FIG. 12 is a pattern diagram showing setting of the area of interest in the object information acquiring apparatus according to the third embodiment. FIG. 12 shows a movement locus 277 of the emission unit 105, which is determined on the basis of the scannable range 171 and an area of interest 275. The operator may determine the setting location of the area of interest 275 by observing the shape and so on of the object 123. In this case, the setting location of the area of interest 275 is dependent on the shape and so on of the object 123. The emission unit 105 emits light beams in order to form an image of the area of interest 275, and therefore the light emission positions are also dependent on the area of interest 275. Accordingly, the emission unit 105 may move in a movement pattern corresponding to the area of interest 275. In other words, the manner in which the emission unit 105 is driven and the processing performed by the area of interest setting unit 271 are determined in accordance with the shape and so on of the object 123. Hence, an infinite number of patterns exist in relation to the manner in which the emission unit 105 is driven and the processing performed by the area of interest setting unit 271, depending on individual differences and the like in the shape and so on of the object 123. Note that the area of interest setting unit 271 may set the area of interest 275 in a substantially cubic shape relative to the object 123, or in other words such that a projection of the area of interest 275 onto the XY plane in FIG. 12 is substantially square.

In this case, rather than storing variation information in the variation storage unit 263 in advance in relation to all of the light emission positions of the emission unit 105, it is preferable to store variation information relating only to a plurality of representative light emission positions, similarly to the second embodiment. The interpolation unit 259 may then generate the variation information relating to the actual light emission positions of the emission unit 105 through interpolation using the variation information stored in the variation storage unit 263.

As a result, photoacoustic measurement can be performed easily in relation to the infinite patterns described above.

The area of interest setting unit 271 receives a command from the operator via the monitor. The present invention is not limited to this configuration, however, and the shape and so on of the object 123 may be acquired automatically by the apparatus 3000 such that a command is output to the area of interest setting unit 271 on the basis of the acquisition result. For example, the shape and so on of the object 123 may be photographed using a solid state imaging device such as a CCD image sensor provided in the apparatus 3000, and a signal based on the imaging result may be transmitted to the area of interest setting unit 271 as the command.

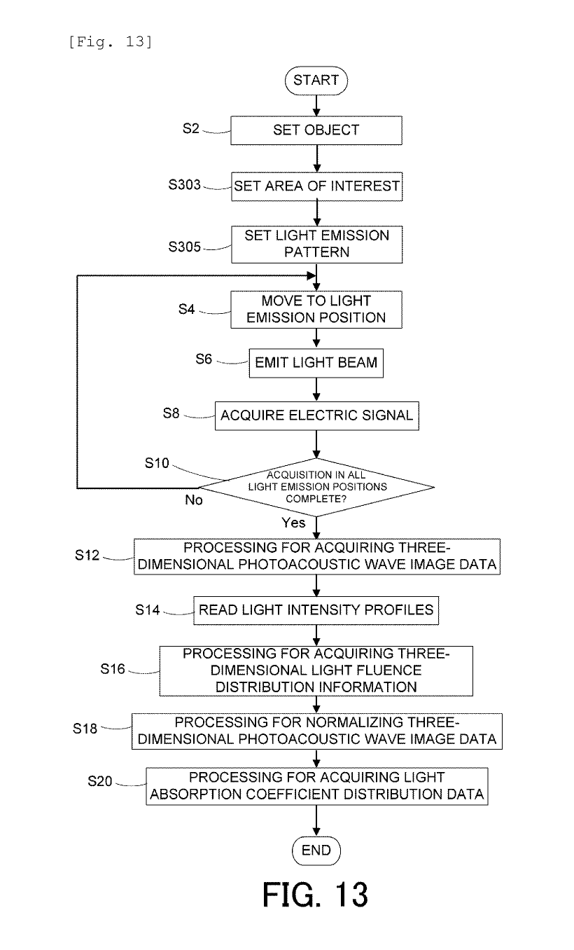

FIG. 13 is a flowchart showing functions of the apparatus 3000 according to the third embodiment. Processing other than that of steps S303 and S305 is identical to the flow shown in FIG. 6A, and will not therefore be described. More specifically, when step S2, which is identical to the processing of FIG. 6A, is complete, the flow advances to step S303. In step S303, the area of interest 275 is set in relation to the object 315 (also including the periphery thereof) by the area of interest setting unit 271. In this case, the area of interest 275 is set by an operator of the apparatus 3000 by controlling the area of interest setting unit 271 manually via an attached monitor (not shown). The flow then advances to step S305.

In step S305, a light emission pattern is set by the operator by inputting the light emission pattern manually on the attached monitor. The light emission pattern is preferably large enough to enable image reconstruction of the entire area of interest 275. In this case, the locus 277 on which the emission unit 105 is to move is determined as the light emission pattern on the basis of the scannable range 171 and the area of interest 275. More specifically, as shown in FIG. 12, the locus 277 is set such that the center of the locus of the spiral movement of the emission unit 105 substantially matches the center of the area of interest 275, and such that the majority of the locus of the spiral movement is enclosed within a projection of the area of interest 275 shown in FIG. 12 onto the XY plane. Thus, image reconstruction can be performed on the entire area of interest 275 easily. Subsequent processing is identical to that shown in FIG. 6A.

### Fourth Embodiment

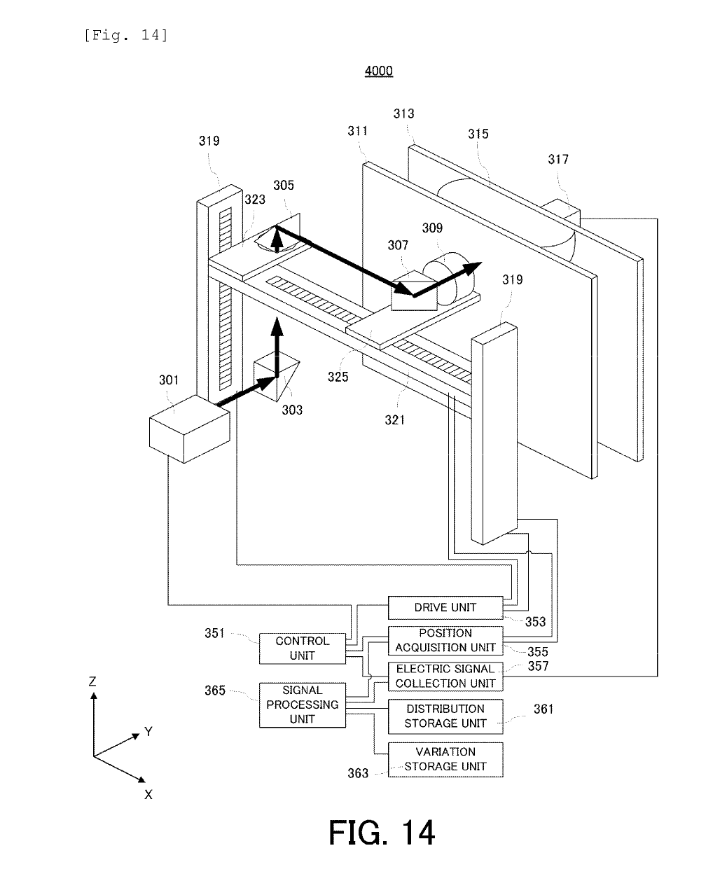

FIG. 14 is a block diagram showing a fourth embodiment of the object information acquiring apparatus according to the present invention. In an object information acquiring apparatus 4000 (abbreviated hereafter to “the apparatus 4000”) according to this embodiment, a space transmission system can be employed as a light transmission unit. In the apparatus 4000, a light source 301, prisms 303, 305, 307 constituting a space transmission system, an emission unit 309, holding plates 311, 313, an acoustic wave reception element 317, a Z axis stage 319, an X axis stage 321, holding units 323, 325, and so on are formed on a base.

The prisms 303, 305, 307 serve as means for bending the advancement direction of a light pulse generated by the light source 301 by 90 degrees. The Z axis stage 319 is configured to be capable of moving the X axis stage in a vertical direction (a Z direction). The holding plates 311, 313 form a pair of plates configured to be capable of holding an object 315 (a breast, for example). The acoustic wave reception element 317 may be formed by arranging piezo elements or the like (note that the present invention is not limited thereto, and any element capable of receiving an acoustic wave may be used) two-dimensionally. Further, at least a part of a probe may serve as the acoustic wave reception element 317. The acoustic wave reception element 317 may be moved in a two-dimensional direction (an XZ plane direction) by the X axis stage 321 and the Z axis stage 319, and may be moved either synchronously with the emission unit 309 or so as to follow the movement of the emission unit 309. The present invention is not limited to this configuration, however, and instead of moving, the acoustic wave reception element 317 may be configured such that the orientation of the direction (the directivity) having the highest reception sensitivity can be varied. Alternatively, a plurality of acoustic wave reception elements 317 may be arranged two-dimensionally in fixed positions. The support unit 323 may be provided with a hole through which the light pulse from the light source 301 can pass so that the light pulse is not blocked. The support unit 325 supports the prism 307 and the emission unit 309, and is configured to be moved in a horizontal direction by the X axis stage 321.

The control unit 351 includes a light source control unit, a drive control unit, a collection control unit, and a system control unit for controlling the other units (none of which are shown in the drawing). The light source control unit controls the light source 301 to generate the light pulse at a desired timing. The light source control unit controls the light source 101 so that the light source 101 generates the light pulse at a repetition frequency of 10 Hz. The drive control unit controls a drive unit 353 so that the drive unit 353 applies a desired movement to the emission unit 309. Further, the drive control unit issues a command to a position acquisition unit 355 so that the position acquisition unit 355 acquires information indicating positions of the X axis stage 321 and the Z axis stage 319 on the basis of the command. The position information may correspond to information relating to the light emission positions of the emission unit 309. The position information may be acquired by the position acquisition unit 355 on the basis of information indicating amounts by which the X axis stage 321 and the Z axis stage 319 have been driven. Alternatively, predetermined light emission position information set in advance may be stored in a memory or the like, and the position information may be acquired on the basis of a light emission timing by referring to the stored light emission position information.

The collection control unit issues a command to an electric signal collection unit 357 to collect signals reaching the acoustic wave reception element 317 over a period extending from a time 0 μsec to a time 50 μsec, where the time 0 μsec is the time at which the light pulse is emitted onto the object 315. Acoustic waves acquired over the period extending from the time 0 μsec to the time 50 μsec are generated in respective positions from the surfaces of the holding plate 311 and the object 315 up to a depth of more than 70 mm in the interior of the object 315.

A distribution storage unit 361 stores information relating to the light intensity profile of the beam section. The information relating to the light intensity distribution may be data acquired as follows. Specifically, when the emission unit 309 is placed in the center of a movable range thereof and a screen is disposed on the side of the holding plate 311 on which the object 315 is positioned, the light intensity profile of a cross-section of a light beam emitted onto the screen by the emission unit 309 is measured. Digital or analog data acquired on the basis of the measurement result may then be used as the information relating to the light intensity distribution.

A variation storage unit 363 stores variation information in relation to each light emission position of the emission unit 309. The variation information may include spreading of the light intensity profile of the beam section caused by a difference in a propagation distance of the light beam during scanning, for example. Alternatively, the variation information may include information indicating an amount of positional deviation in the light intensity profile of the beam section caused by errors occurring when the prisms 303, 305, 307 are attached.

A signal processing unit 365 generates a three-dimensional photoacoustic wave image (apart of the object information) of the interior of the object 315 in each light emission position of the emission unit 309, or in other words in each acoustic wave acquisition position, from the electric signals based on the photoacoustic waves collected by the electric signal collection unit 357 using a UBP algorithm. The signal processing unit 365 then generates information indicating the three-dimensional light fluence distribution of the interior of the object 315 in each light emission position of the emission unit 309 using an optical diffusion equation on the basis of the information relating to the light intensity profiles of the beam sections, stored in the distribution storage unit 361, and the variation information stored in the variation storage unit 363. The three-dimensional light fluence distribution information serves as a part of the object information. Further, the signal processing unit 365 acquires the light absorption coefficient distribution of the interior of the object 315 in each light emission position of the emission unit 309 by normalizing the generated photoacoustic wave images using the three-dimensional light fluence distribution information. The signal processing unit 365 implements these processes over the entire scanning range of the emission unit 309, and finally superimposes the acquired photoacoustic wave images and the light absorption coefficient distributions, whereby a three-dimensional photoacoustic wave image and a light absorption coefficient distribution of the entire object 315 are acquired.

As a result, the three-dimensional light absorption coefficient distribution of the entire object 315 can be acquired with a high degree of precision.

### Fifth Embodiment

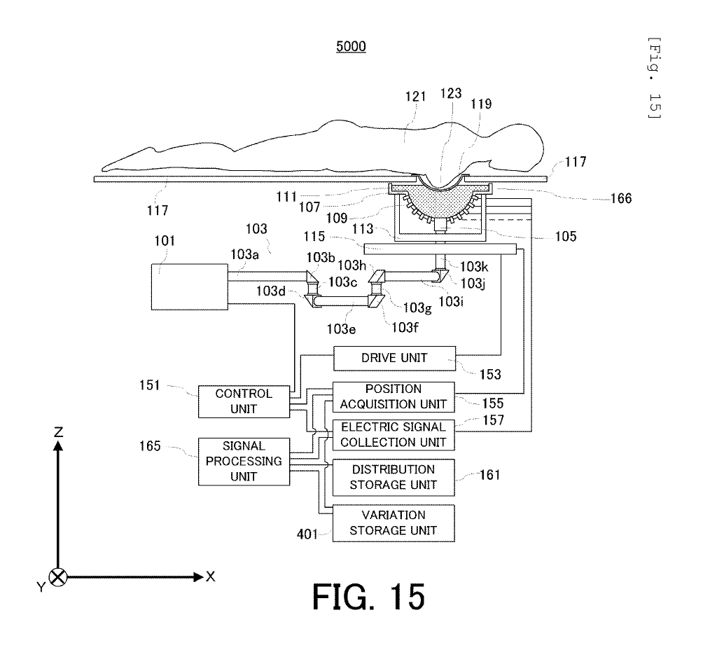

FIG. 15 is a block diagram showing a fifth embodiment of the object information acquiring apparatus according to the present invention. Parts corresponding to FIG. 1, FIG. 6, or FIG. 9 have been allocated identical reference numerals, and description thereof has been omitted when not required. In an object information acquiring apparatus 5000 (abbreviated hereafter to “the apparatus 5000”) according to this embodiment, a relational expression expressing a relationship between the light emission position of the emission unit 105 and the variation information is acquired in advance, and the variation information is generated using this relational expression. On this point, the apparatus 5000 differs from the apparatus 1000 according to the first embodiment, in which the variation information is stored in relation to all of the predetermined light emission positions of the emission unit. The apparatus 5000 includes a variation information generation unit 401, and the variation information generation unit 401 includes the relational expression expressing the relationship between the light emission position (the coordinates) of the emission unit 105 and the variation information. The variation information generation unit 401 generates the variation information on the basis of the information relating to the light emission position of the emission unit 105, which is acquired by the position acquisition unit 155.

The relational expression expressing the relationship between the position of the emission unit 105 and the variation information will be described below.

As described using FIGS. 2A to 2C, the light intensity profile of the beam section in the vertical waveguide 103c has a mirror image relationship with the light intensity profile of the beam section in the vertical waveguide 103g when the cross-section of the horizontal waveguide 103e is used as the plane of symmetry. An incline of this plane of symmetry on the XY plane is determined according to an angle between a lengthwise direction of a projection of the horizontal waveguide 103a onto the XY plane and a lengthwise direction of a projection of the horizontal waveguide 103e onto the XY plane. Each arm of the articulated arm 103 has a set length. Therefore, the incline of the plane of symmetry on the XY plane is determined univocally in accordance with the position of the emission unit 105. Accordingly, the rotation angle of the light intensity profile of the beam section can be defined as a function of the light emission position of the emission unit 105. Further, the amount of positional deviation in the center of gravity of the light intensity profile of the beam section can be acquired as follows. First, an amount of deviation between an optical axis of the concave lens provided in the emission unit 105 and the center of gravity of the actual emitted light beam and a magnification of the emission unit 105 are determined by experiment. Hence, as long as the rotation angle of the light intensity profile of the beam section can be determined, the amount of positional deviation in the center of gravity of the light intensity profile of the beam section can be calculated on the basis of predetermined amounts. The predetermined amounts are the magnification, the amount of deviation from the center of gravity of the actual emitted light beam, and the rotation angle of the light intensity profile of the beam section. Accordingly, the amount of positional deviation in the light intensity profile of the beam section and the rotation angle of the light intensity profile of the beam section can be defined as a function of the light emission position of the emission unit 105. The variation information generation unit 401 may hold the function of the light emission position as the relational expression, receive information indicating the light emission position of the emission unit 105, and generate the variation information on the basis of the relational expression and the light emission position information. Note that the amount of information processed by the variation information generation unit 401 is smaller than that of the signal processing unit 165, and therefore the signal processing unit 165 may be configured to include the functions of the variation information generation unit 401.

As a result, the three-dimensional light absorption coefficient distribution of the entire object 123 can be acquired with a high degree of precision.

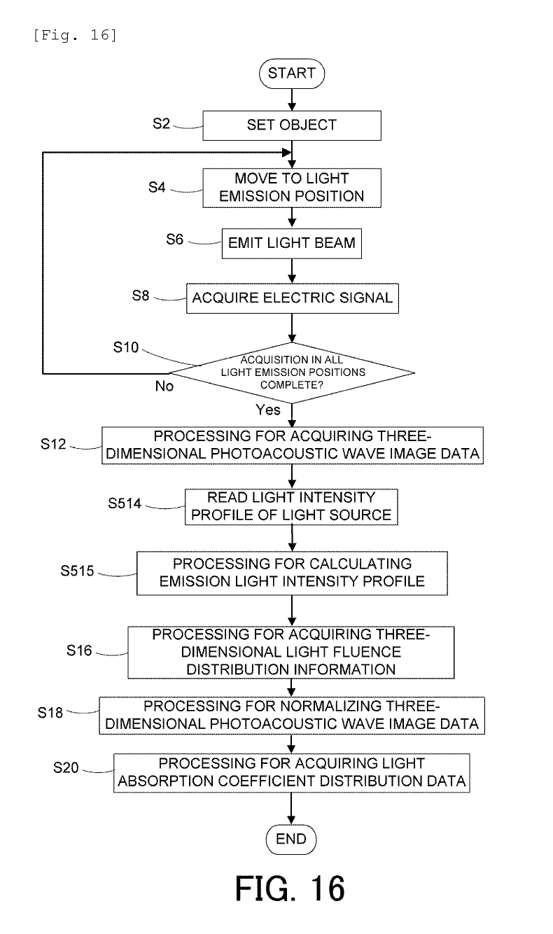

FIG. 16 is a flowchart showing functions of the apparatus 5000 according to the fifth embodiment. Processing other than that of steps S514 and S515 is identical to the flow shown in FIG. 6A, and will not therefore be described. More specifically, when the processing of step S2 to step S12, which is identical to the processing in FIG. 6A, is complete, the flow advances to step S514. In step S514, the light intensity profile of the beam section of the light beam generated by the light source 101 is read from the distribution storage unit 161, whereupon the flow advances to step S515.

In step S515, the light emission position of the emission unit 105 is input into the variation information generation unit 401 as the light emission position information. The light intensity profile of the beam section of the light beam generated by the light source 101, read in step S514, is also input into the variation information generation unit 401. The variation information generation unit 401 then performs calculation processing corresponding to the relational expression using the input light emission position information and light intensity profile as the parameters of the relational expression. A calculation result is acquired as the light intensity profile of the beam section of the light beam emitted from the emission unit 105. Once this processing has been executed in relation to all of the light emission positions, the flow advances to step S16. Subsequent processing is identical to that of FIG. 6A.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-032153, filed on Feb. 20, 2015, which is hereby incorporated by reference herein in its entirety.

### REFERENCE SIGNS

101 light source
103 articulated arm
105 emission unit
161 distribution storage unit
165 signal processing unit
166 acoustic wave reception unit

1. An object information acquiring apparatus comprising:
a light emission unit configured to emit light beams from a plurality of emission positions;
a conversion unit configured to convert acoustic waves generated when an object is irradiated with the light beams emitted by the light emission unit into electric signals;
a beam profile acquisition unit configured to acquire information relating to beam profiles of the light beams emitted by the light emission unit, the beam profiles corresponding respectively to the plurality of emission positions; and
a characteristic information acquisition unit configured to acquire characteristic information of the object on the basis of the information relating to the beam profiles corresponding to the plurality of emission positions and the electric signals.

2. The object information acquiring apparatus according to claim 1, wherein the characteristic information acquisition unit is configured to:
acquire information relating to respective light fluence distributions of the light beams emitted from the plurality of emission positions in an interior of the object by using the information relating to the beam profiles corresponding to the plurality of emission positions; and
acquire the characteristic information of the object on the basis of the information relating to the light fluence distributions and the electric signals.

3. The object information acquiring apparatus according to claim 1, wherein the light emission unit includes:
a light source;
an emission optical system configured to guide a light beam from the light source to the object; and
a moving unit configured to move the emission optical system to the plurality of emission positions.

4. The object information acquiring apparatus according to claim 3, wherein the emission optical system is configured to guide the light beam generated by the light source from the light source to an emission end of the emission optical system while varying an advancement direction thereof, a beam profile of a cross-section of the light beam varying in accordance with the advancement direction.

5. The object information acquiring apparatus according to claim 3, wherein the emission optical system is formed from:
a plurality of waveguides configured to have hollow interiors so that a light beam can propagate therethrough; and
a plurality of joints that connect the plurality of waveguides and include mirrors used to bend a propagation direction of the light beam, and
wherein at least a part of the plurality of joints is configured to rotate about the waveguides connected to the corresponding joints.

6. The object information acquiring apparatus according to claim 3, wherein a shape of the emission optical system is determined in accordance with the emission positions, and
the beam profiles of the light beams emitted in the plurality of emission positions are determined in accordance with the shape of the emission optical system.

7. The object information acquiring apparatus according to claim 1, further comprising a storage unit in which the information relating to the beam profiles is stored,
wherein the beam profile acquisition unit is configured to acquire the information relating to the beam profiles corresponding to the plurality of emission positions by referring to the information relating to the beam profiles, the information being stored in the storage unit.

8. The object information acquiring apparatus according to claim 7, wherein the information relating to the beam profiles corresponding to the plurality of emission positions is stored in the storage unit, and
the beam profile acquisition unit is configured to acquire the information relating to the beam profiles corresponding to the plurality of emission positions by reading the information relating to the beam profiles corresponding to the plurality of emission positions, the information being stored in the storage unit.

9. The object information acquiring apparatus according to claim 7, wherein the beam profile acquisition unit is configured to acquire the information relating to the beam profiles corresponding to the plurality of emission positions by interpolating the information relating to the beam profiles, the information being stored in the storage unit.

10. The object information acquiring apparatus according to claim 1, further comprising a storage unit in which a relational expression or a relationship table expressing relationships between the emission positions and the beam profiles is stored,
wherein the beam profile acquisition unit is configured to acquire the information relating to the beam profiles corresponding to the plurality of emission positions by referring to the relational expression or the relationship table stored in the storage unit.

11. The object information acquiring apparatus according to claim 1, further comprising a storage unit in which a relational expression or a relationship table expressing relationships between the emission positions and amounts of variation in the beam profiles is stored,
wherein the beam profile acquisition unit is configured to acquire the information relating to the beam profiles corresponding to the plurality of emission positions by referring to the relational expression or the relationship table stored in the storage unit.

12. The object information acquiring apparatus according to claim 11, wherein the beam profile acquisition unit is configured to acquire information relating to a reference beam profile corresponding to a reference emission position, and acquire the information relating to the beam profiles corresponding to the plurality of emission positions on the basis of the reference beam profile and the relational expression or the relationship table stored in the storage unit.

13. The object information acquiring apparatus according to claim 1, further comprising a position acquisition unit configured to acquire information relating to the emission positions in which the light beams are emitted from the light emission unit,
wherein the beam profile acquisition unit is configured to acquire the information relating to the beam profiles corresponding to the plurality of emission positions using the information relating to the emission positions.

14. The object information acquiring apparatus according to claim 1, wherein the acoustic wave reception unit is configured to hold a matching agent through which the acoustic waves from the object propagate to the conversion unit.

15. The object information acquiring apparatus according to claim 1, wherein the conversion unit includes a plurality of acoustic wave reception elements.

16. The object information acquiring apparatus according to claim 15, wherein the plurality of acoustic wave reception elements are disposed such that respective directions of the plurality of acoustic wave reception elements in which a reception sensitivity is highest are gathered together.

17. A signal processing method for acquiring characteristic information of an object using electric signals derived from acoustic waves that are generated when the object is irradiated with light beams emitted from a plurality of emission positions, comprising the steps of:
acquiring information relating to beam profiles of the light beams emitted from the plurality of emission positions, the beam profiles corresponding respectively to the plurality of emission positions; and
acquiring the characteristic information on the basis of the information relating to the beam profiles corresponding to the plurality of emission positions and the electric signals.

18. A non-transitory computer readable storing medium recording a computer program for causing a computer to perform a signal processing method for acquiring characteristic information of an object using electric signals derived from acoustic waves that are generated when the object is irradiated with light beams emitted from a plurality of emission positions, comprising the steps of:
acquiring information relating to beam profiles of the light beams emitted from the plurality of emission positions, the beam profiles corresponding respectively to the plurality of emission positions; and
acquiring the characteristic information on the basis of the information relating to the beam profiles corresponding to the plurality of emission positions and the electric signals.
